(19) United States
(12) Patent Application Publication
(10) Pub. No.: US 2020/0345694 A1
(43) Pub. Date: Nov. 5, 2020

(54) ANTIAGING COMPOSITION COMPRISING CYTOCHALASIN D OR SAG, AND METHOD FOR SCREENING ANTIAGING SUBSTANCE

(71) Applicant: AMOREPACIFIC CORPORATION, Seoul (KR)

(72) Inventors: Hyun Jung CHOI, Yongin-si (KR); Hyoung June KIM, Yongin-si (KR); Ji Yong JUNG, Yongin-si (KR); Tae Ryong LEE, Yongin-si (KR)

(73) Assignee: AMOREPACIFIC CORPORATION, Seoul (KR)

(21) Appl. No.: 16/759,211

(22) PCT Filed: Jul. 24, 2018
(86) PCT No.: PCT/KR2018/008344

§ 371 Date: Jul. 9, 2020

(30) Foreign Application Priority Data
Oct. 27, 2017 (KR) 10-2017-0141208

(51) Int. Cl. A61K 31/4035; A61K 31/4436; A61K 36/06; A61K 8/49; A61K 8/9728; A23L 33/10; A61Q 19/08

(52) U.S. Cl. CPC A61K 31/4035; A61K 31/4436; A61K 36/06; A61K 9/0053; A61K 8/9728; A23L 33/10; A61Q 19/08; A61K 8/492

(57) ABSTRACT

The present disclosure relates to an antiaging composition containing cytochalasin D or SAG, and a method for screening an antiaging substance, and provides a new antiaging substance and a new method for discovering a new antiaging substance. Thus, it is possible to greatly contribute to the development of beauty industries and to meeting demands related with beauty and antiaging, if the present disclosure is effectively used.

### TECHNICAL FIELD

The present disclosure relates to an antiaging composition comprising cytochalasin D or SAG, and a method for screening an antiaging substance.

### BACKGROUND ART

Recently, as the average life expectancy is increased gradually, it becomes important how long youth can be maintained rather than how long one can live. Aging is an inevitable process. In the modern society where environmental pollution and stress are getting worse and the frequency of exposure to UV increases, the aging begins in earlier years than before.

The aging of the organs of the human body proceeds gradually at certain rates. In particular, skin aging proceeds through complicated processes as various natural aging processes are influenced by the external environment. Mostly, skin aging begins with dryness of the skin, specifically with decreased skin elasticity and wrinkle formation due to lack of moisture. As one grows older, the skin loses elasticity due to decreased cellular regenerative ability and wrinkles are formed as the connective tissues of the skin become loose due to hormonal and UV factors. The specific phenomena of skin aging include skin roughening due to decreased skin regeneration and accumulation of aged corneocytes, wrinkle formation due to decreased collagen synthesis, pigmentation such as liver spots, age spots, etc. due to decreased protection from UV, and ready response to drugs or irritation due to decreased skin-protecting function.

The primary cilium is an organelle projecting from the cell body. It is known as a sensory organelle that senses various external stimuli. But, nothing is known about the antiaging effect of the primary cilium.

### DISCLOSURE

### Technical Problem

The present disclosure is directed to a new substance capable of improving skin aging and a new method for screening the substance.

### Technical Solution

The present disclosure provides a skin antiaging composition, which contains cytochalasin D, 3-chloro-N-[trans-4-(methylamino)cyclohexyl]-N-[[3-(4-pyridinyl)phenyl]methyl]benzo[b]thiophene-2-carboxamide, an optical isomer or stereoisomer thereof, a pharmaceutically acceptable salt thereof, a hydrate thereof, or a solvate thereof as an active ingredient.

In addition, the present disclosure provides a method for screening a substance with skin antiaging effect, which includes: a step of treating skin cells with a test substance; and a step of identifying the relative degree of formation of primary cilia in the skin cells before and after the treatment with the test substance.

In addition, the present disclosure provides a kit for screening a substance with skin antiaging effect, which includes a measurement unit indicating the relative degree of formation of primary cilia before and after treatment with a test substance, and a method for providing information required for diagnosis of skin condition, which includes a step of identifying the degree of formation of primary cilia.

### Advantageous Effects

Since the present disclosure provides a new antiaging composition and a new method for screening a new antiaging substance, it is possible to greatly contribute to the development of beauty industries and to meeting demands related with beauty and antiaging, if the present disclosure is utilized effectively.

### BRIEF DESCRIPTION OF DRAWINGS

FIG. 1 shows the expression pattern of primary cilia in fibroblasts treated with cytochalasin D or SAG.

FIG. 2 shows a result of identifying the expression pattern of procollagen in fibroblasts treated with cytochalasin D or SAG by western blot.

### BEST MODE

In the present disclosure, “skin” refers to the tissue covering the body surface of an animal. The term is used in the broadest concept, including not only the tissue covering the surface of face, body, etc., but also the scalp and hair.

In the present disclosure, “cytochalasin D” may also be expressed as Cyto D.

In the present disclosure, “3-chloro-N-[trans-4-(methylamino)cyclohexyl]-N-[[3-(4-pyridinyl)phenyl]methyl]benzo [b]thiophene-2-carboxamide” may also be expressed as an Smo agonist, a

Smoothened agonist, or SAG. The 3-chloro-N-[trans-4-(methylamino)cyclohexyl]-N-[[3-(4-pyridinyl)phenyl]methyl]benzo[b]thiophene-2-carboxamide may be a substance inducing the expression of the Smoothened protein which plays a key role in the hedgehog signaling pathway.

In the present disclosure, an “isomer” includes, in particular, not only an optical isomer (e.g., an essentially pure enantiomer, an essentially pure stereoisomer or a mixture thereof) or an essentially pure diastereomer, but also a conformational isomers (i.e., an isomer differing only in the angle of one or more chemical bond), a structural isomer, a position isomer (particularly, a tautomer) or a geometric isomer (e.g., a cis-trans isomer).

In the present disclosure, “essentially pure” means, when used with regard to an enantiomer, stereoisomer or diastereomer, that a specific compound, e.g., an enantiomer, stereoisomer or diastereomer, is present in an amount of about 90% (w/w) or more, specifically about 95% or more, more specifically about 97% or more or about 98% or more, further more specifically about 99% or more, even more specifically about 99.5% or more.

In the present disclosure, “acceptable” means being devoid of substantial toxic effect when used in a usually employed medicinal dosage and thereby being approvable or approved by the government or a regulatory organization comparable thereto or being listed or described in the food code, functional health food code or other general pharmacopoeias for use in animals, more particularly in human.

In the present disclosure, an “acceptable salt” refers to a salt according to an aspect of the present disclosure that is generally, medicinally or pharmaceutically acceptable and possesses the desired activity of a parent compound. The salt includes: (1) an acid addition salt formed from an inorganic acid such as hydrochloric acid, hydrobromic acid, sulfuric acid, nitric acid, phosphoric acid, etc., or formed from an organic acid such as acetic acid, propionic acid, hexanoic acid, cyclopentylpropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfuric acid, gluconic acid, glutamic acid, hydroxynaphthoic acid, salicylic acid, stearic acid and muconic acid; or (2) a salt formed when an acidic proton present in the parent compound is replaced.

In the present disclosure, a “hydrate” refers to a compound to which water is bonded. The term is used in a broad sense, including an inclusion compound which lacks chemical bonding with water.

In the present disclosure, a “solvate” refers to a higher-order compound formed between a molecule or ion of solute, and a molecule or ion of solvent.

In the present disclosure, the “primary cilium” (primary cilia in plural) refers to an organelle projecting from the cell body, which senses various external stimuli including chemical stimulation, physical stimulation, light, osmotic pressure, fluid flow, gravity, etc.

In the present disclosure, a “relative degree of formation” may mean a degree indicating the occurrence of formation or expression of primary cilia or change in the number, quantity, or quality of formation or expression of primary cilia, when comparing the degree of formation or expression of primary cilia in cells subjected to a certain condition or not. The degree of formation may, for example, mean the number or quantity of the cells that form or express primary cilia.

In the present disclosure, a “substance with skin antiaging effect” collectively refers to a substance which prevents, improves or treats the aging phenomena of the whole body or a specific organ (e.g., skin) of the body, and may include a compound, a polymer, a DNA, an RNA, a peptide, a protein, etc., although not being limited thereto.

In an aspect, the present disclosure provides a skin antiaging composition containing, as a substance increasing the expression of primary cilia, cytochalasin D, 3-chloro-N-[trans-4-(methylamino)cyclohexyl]-N-[[3-(4-pyridinyl)phenyl]methyl]benzo[ b]thiophene-2-carboxamide (SAG), an optical isomer or stereoisomer thereof, a pharmaceutically acceptable salt thereof, a hydrate thereof or a solvate thereof as an active ingredient.

In another aspect, the present disclosure provides a method for preventing, treating or improving one or more selected from a group consisting of procollagen reduction, skin wrinkling, skin elasticity, skin regeneration, skin volume, scar and old look; or a method for skin antiaging, the method comprising: administering cytochalasin D, 3-chloro-N-[trans-4-(methylamino)cyclohexyl]-N-[[3-(4-pyridinyl)phenyl]methyl]benzo[b]thiophene-2-carboxamide, an optical isomer or stereoisomer thereof, a pharmaceutically acceptable salt thereof, a hydrate thereof or a solvate thereof to a subject as a substance increasing the expression of primary cilia.

In an aspect, the present disclosure provides a use of cytochalasin D, 3-chloro-N-[trans-4-(methylamino)cyclohexyl]-N-[[3-(4-pyridinyl)phenyl]methyl]benzo[b]thiophene-2-carboxamide, an optical isomer or stereoisomer thereof, a pharmaceutically acceptable salt thereof, a hydrate thereof or a solvate thereof as a substance increasing the expression of primary cilia for preparation of a composition for preventing, treating or improving one or more selected from a group consisting of procollagen reduction, skin wrinkling, skin elasticity, skin regeneration, skin volume, scar and old look; or skin antiaging.

In an aspect, the present disclosure provides cytochalasin D, 3-chloro-N-[trans-4-(methylamino)cyclohexyl]-N-[[3-(4-pyridinyl)phenyl]methyl]benzo[b]thiophene-2-carboxamide, an optical isomer or stereoisomer thereof, a pharmaceutically acceptable salt thereof, a hydrate thereof or a solvate thereof as an active ingredient for increasing the expression of primary cilia for use in preventing, treating or improving one or more selected from a group consisting of procollagen reduction, skin wrinkling, skin elasticity, skin regeneration, skin volume, scar and old look; or skin antiaging.

In an aspect, the present disclosure provides a non-therapeutic, cosmetic use of cytochalasin D, 3-chloro-N-[trans-4-(methylamino)cyclohexyl]-N-[[3-(4-pyridinyl)phenyl]methyl]benzo[b]thiophene-2-carboxamide, an optical isomer or stereoisomer thereof, a pharmaceutically acceptable salt thereof, a hydrate thereof or a solvate thereof as an active ingredient for increasing the expression of primary cilia for preventing, treating or improving one or more selected from a group consisting of procollagen reduction, skin wrinkling, skin elasticity, skin regeneration, skin volume, scar and old look; or skin antiaging.

In an exemplary embodiment, the cytochalasin D may be an actin polymerization inhibitor and may be an alkaloid with the formula C₃₀H₃₇NO₆. It may be represented by Chemical Formula 1.

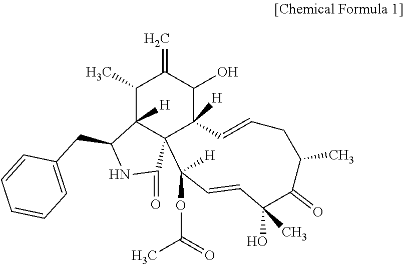

In another exemplary embodiment, the cytochalasin D may be derived from a mold. Alternatively, the cytochalasin D may be obtained through synthesis, processed from another substance or derived from other organisms or microorganisms.

In an exemplary embodiment, the 3-chloro-N-[trans-4-(methylamino)cyclohexyl]-N-[[3-(4-pyridinyl)phenyl]methyl]benzo[b]thiophene-2-carboxamide may be a chlorobenzothiophene-based compound with the formula C₂₈H₂₈ClN₃OS. It may be represented by Chemical Formula 2.

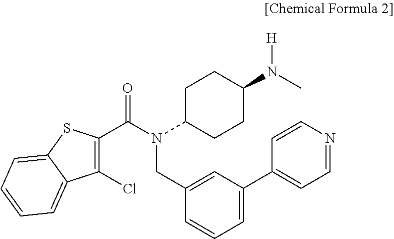

The compound may be obtained through synthesis, processed from another substance or derived from organisms or microorganisms.

In another exemplary embodiment, the mold may be Zygosporiurn mansoni.

In another exemplary embodiment, the aging may be caused by reduction of collagen or procollagen.

In another exemplary embodiment, the active ingredient may enhance the synthesis of collagen or procollagen.

In another exemplary embodiment, the skin antiaging may be prevention, treatment or improvement of one or more selected from a group consisting of skin wrinkling, skin elasticity, skin regeneration, skin volume, scar and old look. In an exemplary embodiment, the composition may be for preventing old look, and the prevention of old look includes prevention of old for beauty care and therapeutic purposes.

According to an aspect of the present disclosure, after treatment with the active ingredient, the expression of primary cilia is increased in fibroblasts and, accordingly, the expression of collagen or procollagen is also increased as compared to before the treatment with the active ingredient. Through this, it can be seen that the active ingredient may be used as an active ingredient of an antiaging composition. In addition, the number of cells with increased expression of primary cilia is also increased after treatment with the active ingredient. Also through this, it can be seen that the active ingredient may be used as an active ingredient of an antiaging composition.

In another exemplary embodiment, the composition may be administered by transdermal administration or oral administration.

In another exemplary embodiment, the administration dosage of the active ingredient may be 0.0001-20 mg/kg/day. In an exemplary embodiment, the administration dosage may be 0.0001 mg/kg/day or more, 0.0005 mg/kg/day or more, 0.001 mg/kg/day or more, 0.005 mg/kg/day or more, 0.01 mg/kg/day or more, 0.05 mg/kg/day or more, 0.1 mg/kg/day or more, 0.5 mg/kg/day or more, 0.8 mg/kg/day or more, 1 mg/kg/day or more, 2 mg/kg/day or more, 3 mg/kg/day or more, 5 mg/kg/day or more, 8 mg/kg/day or more, 10 mg/kg/day or more, 12 mg/kg/day or more, 15 mg/kg/day or more, or 18 mg/kg/day or more. In addition, the administration dosage may be 20 mg/kg/day or less, 18 mg/kg/day or less, 15 mg/kg/day or less, 12 mg/kg/day or less, 10 mg/kg/day or less, 8 mg/kg/day or less, 5 mg/kg/day or less, 3 mg/kg/day or less, 2 mg/kg/day or less, 1 mg/kg/day or less, 0.8 mg/kg/day or less, 0.5 mg/kg/day or less, 0.1 mg/kg/day or less, 0.05 mg/kg/day or less, 0.01 mg/kg/day or less, 0.005 mg/kg/day or less, 0.001 mg/kg/day or less, or 0.0005 mg/kg/day or less.

In another exemplary embodiment, the content of the active ingredient may be 0.0001-20 wt % based on the total weight of the composition. In an exemplary embodiment, the content may be 0.0001 wt % or higher, 0.0005 wt % or higher, 0.001 wt % or higher, 0.005 wt % or higher, 0.01 wt % or higher, 0.05 wt % or higher, 0.1 wt % or higher, 0.3 wt % or higher, 0.5 wt % or higher, 0.8 wt % or higher, 1 wt % or higher, 3 wt % or higher, 5 wt % or higher, 8 wt % or higher, 10 wt % or higher, 12 wt % or higher, 15 wt % or higher, or 18 wt % or higher based on the total weight of the composition. In addition, the content may be 20 wt % or lower, 18 wt % or lower, 15 wt % or lower, 12 wt % or lower, 10 wt % or lower, 8 wt % or lower, 5 wt % or lower, 3 wt % or lower, 1 wt % or lower, 0.8 wt % or lower, 0.5 wt % or lower, 0.3 wt % or lower, 0.1 wt % or lower, 0.05 wt % or lower, 0.01 wt % or lower, 0.005 wt % or lower, 0.001 wt % or lower, or 0.0005 wt % or lower based on the total weight of the composition.

In another exemplary embodiment, the composition may be a food, pharmaceutical or cosmetic composition.

The formulation of the food composition is not particularly limited. For example, it may be formulated into a tablet, a granule, a pill, a powder, a liquid such as a drink, a caramel, a gel, a bar, a tea bag, etc. Each formulation of the food composition may contain, in addition to the active ingredient, ingredients that are commonly used in the art and can be selected by those of ordinary skill in the art depending on the particular formulation or purpose of use. A synergistic effect may be achieved when other ingredients are used together. In addition, the food may be a functional health food.

The composition may be administered by various methods such as simple intake, drinking, injection, spraying, squeezing, etc.

In the food composition according to an aspect of the present disclosure, determination of the administration dosage of the active ingredient is within the level of those of ordinary skill in the art and may vary depending on various factors such as the age and health condition of a subject of administration, the presence of complications(s), etc.

The food composition according to an aspect of the present disclosure may be, for example, various foods such as chewing gum, caramel products, candies, ices, confectionery, etc., beverage products such as soft drinks, mineral water, alcoholic beverages, etc., or functional health food products including vitamins, minerals, etc.

Besides, the food composition according to an aspect of the present disclosure may contain various nutrients, vitamin, minerals (electrolytes), flavors such as synthetic flavors and natural flavors, colorants, extenders (cheese, chocolate, etc.), pectic acid and its salts, alginic acid and its salts, organic acids, protective colloidal thickeners, pH control agents, stabilizers, antiseptics, glycerin, alcohols, carbonating agents used in carbonated beverages, etc. In addition, the food composition according to an aspect of the present disclosure may contain a pulp for preparing natural fruit juice, fruit juice beverages and vegetable beverages. These ingredients may be used either independently or in combination. The proportion of these additives is of no great importance. In general, they may be contained in an amount of about 0-60 parts by weight per 100 parts by weight of the composition according to an aspect of the present disclosure.

The cosmetic composition may further contain a functional additive and ingredients contained in common cosmetic compositions. The functional additive may include an ingredient selected from a group consisting of a water-soluble vitamin, an oil-soluble vitamin, a polypeptide, a polysaccharide, a sphingolipid and a seaweed extract. In addition, the cosmetic composition may contain an oil or fat ingredient, a moisturizer, an emollients, a surfactant, an organic or inorganic pigment, an organic powder, a UV absorber, an antiseptic, a sterilizer, an antioxidant, a plant extract, a pH adjuster, an alcohol, a colorant, a fragrance, a blood circulation stimulant, a skin cooler, a deodorant, purified water, etc.

The formulation of the cosmetic composition is not specially limited and may be selected adequately depending on purposes. For example, the composition may be prepared into one or more formulation selected from a group consisting of a skin lotion, a skin softener, a skin toner, an astringent, a lotion, a milk lotion, a moisturizing lotion, a nourishing lotion, a massage cream, a nourishing cream, a moisturizing cream, a hand cream, a foundation, an essence, a nourishing essence, a pack, a soap, a cleansing foam, a cleansing lotion, a cleansing cream, a body lotion and a body cleanser, although not being limited thereto.

In an aspect of the present disclosure, when the formulation is a paste, a cream or a gel, an animal fiber, a plant fiber, a wax, paraffin, starch, tragacanth, a cellulose derivative, polyethylene glycol, silicone, bentonite, silica, talc, zinc oxide, etc. may be used as a carrier ingredient.

In an aspect of the present disclosure, when the formulation is a powder or a spray, lactose, talc, silica, aluminum hydroxide, calcium silicate or polyamide powder may be used as a carrier ingredient. Particularly, a spray may further contain a propellant such as chlorofluorohydrocarbon, propane/butane or dimethyl ether.

In an aspect of the present disclosure, when the formulation is a solution or an emulsion, a solvent, a solubilizer or an emulsifier may be used as a carrier ingredient. Examples include water, ethanol, isopropanol, ethyl carbonate, ethyl acetate, benzyl alcohol, benzyl benzoate, propylene glycol, 1,3-butylene glycol, an aliphatic ester of glycerol, polyethylene glycol or a fatty acid ester of sorbitan.

In an aspect of the present disclosure, when the formulation is a suspension, a liquid diluent such as water, ethanol or propylene glycol, a suspending agent such as ethoxylated isostearyl alcohol, an ester of propoxyethylene sorbitol and an ester of propoxyethylene sorbitan, microcrystalline cellulose, aluminum metahydroxide, bentonite, agar, tragacanth, etc. may be used as a carrier ingredient.

In an aspect of the present disclosure, when the formulation is a surfactant-containing cleanser, an aliphatic alcohol sulfate, an aliphatic alcohol ether sulfate, sulfosuccinic monoester, isethionate, an imidazolinium derivative, methyl taurate, sarcosinate, a fatty acid amide, an ether sulfate, an alkyl amidobetaine, an aliphatic alcohol, a fatty acid glyceride, a fatty acid diethanolamide, a plant oil, a lanolin derivative, an fatty acid ester of ethoxylated glycerol, etc. may be used as a carrier ingredient.

The pharmaceutical composition according to an aspect of the present disclosure may be administered orally, parenterally, rectally, topically, transdermally, intravenously, intramuscularly, intraperitoneally, subcutaneously, etc. Formulations for oral administration may include a tablet, a pill, a soft or hard capsule, a granule, a powder, a fine granule, a liquid, an emulsion or a pellet, although not being limited thereto. Formulations for parenteral administration may include a solution, a suspension, an emulsion, a gel, an injection, a medicinal drip, a suppository, a patch, a spray, etc., although not being limited thereto. These formulations may be prepared easily according to common methods in the art and may further contain a surfactant, an excipient, a wetting agent, an emulsification promoter, a suspending agent, a salt or a buffer for control of osmotic pressure, a colorant, a flavorant, a stabilizer, an antiseptic, a preservative or other commonly used adjuvants. The pharmaceutical composition may be a formulation for external application to skin. The formulation for external application to skin includes any formulation that can be applied externally to the skin and medicinal products or quasi-drug products of various formulations may be included therein.

The composition according to an aspect of the present disclosure may also contain a pharmaceutically acceptable salt. The salt may include: (1) an acid addition salt formed from an inorganic acid such as hydrochloric acid, hydrobromic acid, sulfuric acid, nitric acid, phosphoric acid, etc., or formed from an organic acid such as acetic acid, propionic acid, hexanoic acid, cyclopentylpropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfuric acid, gluconic acid, glutamic acid, hydroxynaphthoic acid, salicylic acid, stearic acid and muconic acid; or (2) a salt formed when an acidic proton present in the parent compound is replaced.

The application amount or administration dosage of the pharmaceutical composition according to an aspect of the present disclosure will vary depending on the age, sex and body weight of a subject of administration, pathological condition and severity thereof, administration route or the discretion of a prescriber. The determination of the administration dosage of the active ingredient based on these factors is within the level of those of ordinary skill in the art.

In another aspect, the present disclosure may provide a method for screening a substance with skin antiaging effect, which includes a step of treating skin cells with a test substance; and a step of identifying the relative degree of formation of primary cilia in the skin cells before and after the treatment with the test substance.

In an exemplary embodiment, the skin cells may be fibroblasts.

In another exemplary embodiment, the method may further include a step of determining the test substance as a substance with skin antiaging effect when the primary cilia formed after the treatment with the test substance are more than the primary cilia before the treatment.

In another exemplary embodiment, the relative degree of formation of primary cilia may be determined through one or more of comparison of the number of the skin cells wherein primary cilia are expressed and comparison of the amount of primary cilia expressed in one cell.

In a specific exemplary embodiment, the relative degree of formation of primary cilia may be identified with a method that can be adequately selected by those skilled in the art, such as immunofluorescence assay, western blot, dot blot, ELISA, northern blot, PCR, RT-qPCR, GC-MS, LC-MS, NMR, etc., although not being limited thereto. In another exemplary embodiment, the relative degree of formation of primary cilia may be identified through fluorescent staining.

In another exemplary embodiment, the relative degree of formation of primary cilia may be identified with the degree of expression of collagen or procollagen.

In another aspect, the present disclosure may provide a kit for screening a substance with skin antiaging effect, which includes a measurement unit indicating the relative degree of formation of primary cilia before and after treatment with a test substance.

In an exemplary embodiment, the kit may further include skin cells and an instruction, and the method described above may be described in the instruction.

In another aspect, the present disclosure may provide a method for providing information required for diagnosis of skin condition, which includes a step of identifying the degree of formation of primary cilia.

In an exemplary embodiment, the primary cilia may be primary cilia of fibroblasts of the whole skin or a specific part of the skin.

In another exemplary embodiment, the skin condition may be skin condition associated with collagen or procollagen. In the present disclosure, the ‘skin condition associated with collagen or procollagen’ refers to skin condition directly or indirectly associated with collagen or procollagen. For example, the skin condition includes skin with high or low sensitivity to a stimulation forming collagen or procollagen, skin with possibility of increased or inhibited synthesis of collagen or procollagen, skin with high or low risk of a skin disease associated with collagen or procollagen, skin with or without aging symptoms, skin with many or few wrinkles, skin with high or low elasticity, skin with high or low regenerative ability, etc.

In another exemplary embodiment, the degree of formation of primary cilia may be identified by comparing the degree of formation of primary cilia before and after a specific stimulation inhibiting the formation of collagen or procollagen is applied. For example, the degree of formation of primary cilia may be measured by extracting fibroblasts from the skin or directly without isolating the cells. The extraction or measurement may be performed by any method well known to those skilled in the art.

In another exemplary embodiment, the diagnosis of skin condition may be determining whether the skin has high sensitivity to a stimulation inhibiting the formation of collagen or procollagen.

In another exemplary embodiment, the stimulation may be one or more of exogenous stimulation and endogenous stimulation.

In another exemplary embodiment, the exogenous stimulation may include ultraviolet radiation, physical pressure, wound, stimulation by a heavy metal, stimulation by a chemical substance, microbial infection and stress.

In another exemplary embodiment, the endogenous stimulation may include change of oxidative stress in vivo, aging, inflammation, hormonal change, dysfunction of a digestive organ including the liver, metabolic dysfunction, nutritional abnormality, and excess or deficiency of vitamins or minerals.

In another exemplary embodiment, the degree of formation of primary cilia may be identified by comparing the degree of formation of primary cilia in the whole skin or a specific part of the skin of a subject at two specific time points. For example, the degree of formation of primary cilia may be measured by extracting fibroblasts from the whole skin or a specific part of the skin of a subject or directly without isolating the cells. The extraction or measurement may be performed by any method well known to those skilled in the art.

In another exemplary embodiment, it may be determined that there is a possibility of developing skin aging when collagen or procollagen is decreased in the whole skin or a specific part of the skin.

In another exemplary embodiment, a grade indicating the possibility of skin aging may be evaluated depending on the degree of formation of primary cilia.

In another exemplary embodiment, the grade may be determined based on a reference value determined from data about the degree of formation of primary cilia obtained from skin samples of a given number of subjects.

Until now, nothing is known about the relationship between aging and the primary cilia in cells. The present disclosure has elucidated the relationship. According to the present disclosure, it has been found out that the synthesis of collagen and procollagen can be enhanced by inducing the formation of primary cilia by treating fibroblasts with a specific substance. That is to say, it has been found out that, when fibroblasts are treated with a substance with skin antiaging effect, the formation of primary cilia is enhanced and, accordingly, the synthesis of collagen and procollagen is enhanced. On the contrary, it has been found out that, when fibroblasts are treated with a substance inducing aging, the formation of primary cilia is inhibited and the synthesis of collagen and procollagen is also inhibited accordingly.

Based on this relationship, the inventors of the present disclosure have found out a method for screening a substance which exhibits an antiaging effect. In addition, a kit including a measurement unit capable of identifying or indicating the degree of formation of primary cilia can be provided by utilizing this feature. Furthermore, the inventors have found out a method for providing information required for evaluation or diagnosis of skin condition associated with collagen or procollagen by identifying the degree of formation of primary cilia.

Hereinafter, the constitution and effect of an aspect of the present disclosure will be described in detail through test examples. However, the following examples are for illustrative purposes only and the scope of the present disclosure is not limited by them.

[Test Example 1 Investigation of Expression of Primary Cilia in Skin Cells

Normal human neonatal dermal fibroblasts were plated in a Lab-Tex™ 2-well glass chamber slide (Thermo Scientific, Waltham, Mass., USA) with 0.5×10⁵ cells per well. 24 hours later, the cells were treated with a test substance (1.5 mL of 0.2 μM cytochalasin D, or 1.5 mL of 2 μM SAG). 48 hours, the cells were immobilized and primary cilia were stained.

The primary cilia were stained with ARL13B (Proteintech) and a-acetylated tubulin (Sigma-Aldrich) antibodies. The cells were incubated overnight at 4° C. with ARL13B antibody diluted to 1/500 based on weight and a-acetylated tubulin antibody diluted to 1/1000 based on weight in a PBS buffer supplemented with 0.1% Triton™ X-100 and 5% normal goat serum. Then, the cells were treated at room temperature for 2 hours with Alexa Fluor 594 goat anti-mouse IgG and 488 goat anti-rabbit IgG (Invitrogen) as secondary antibodies. The nuclei were stained with DAPI and observed under a confocal microscope. The experiment was repeated at least twice for independent test groups.

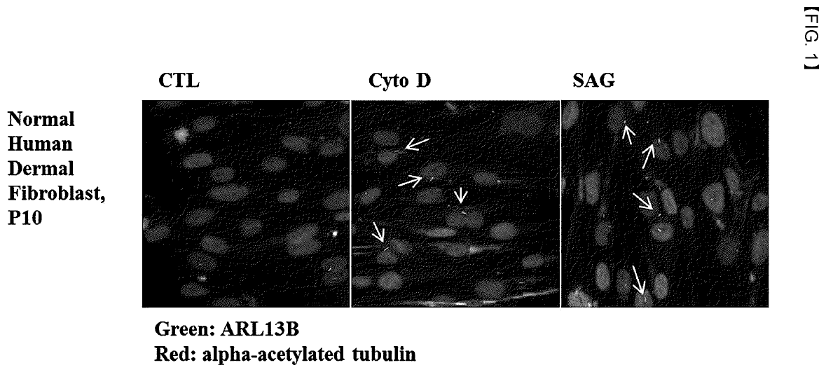

As seen from FIG. 1 ('CTL': control group, ‘Cyto D’: cytochalasin D-treated group, ‘SAG’: SAG-treated group), the bioantenna of fibroblasts, i.e. primary cilia, were increased when the cells were treated with 0.2 μM cytochalasin D or 2 μM SAG. In FIG. 1, the primary cilia are indicated by arrows. The cell nuclei appear as dark gray round regions, and the α-acetylated tubulin appears as a white region. It can be seen that there exists one primary cilium per cell.

[Test Example 2] Investigation of Effect of Preventing Skin Aging

It was investigated whether the increase in primary cilia leads to increased secretion of procollagen to the medium.

Specifically, normal human dermal fibroblasts were cultured in DMEM supplemented with 10% FBS (fetal bovine serum), 100 mg/L streptomycin and 100,000 U/L penicillin. The cultured fibroblasts seeded onto a 6-well plate with 1×10⁵ cells per well and 1.5 mL of a medium containing 0.2 μM cytochalasin D or 2 pM SAG as a test substance was added 24 hours later. This leads to increased expression of primary cilia as in Test Example 1. 48 hours after the treatment with the test substance, the medium was collected and the amount of procollagen I in the medium was measured by western blotting. The procollagen I antibody was purchased from Developmental Studies Hybridoma Bank and diluted in a PBS buffer containing 0.1% Trito™ X-100 and 5% normal goat serum to 1/10000 based on weight. As a control group (loading control), electrophoresed polyacrylamide gel was stained with 0.05% Coomassie blue and then observed. The experiment was repeated at least twice for independent test groups.

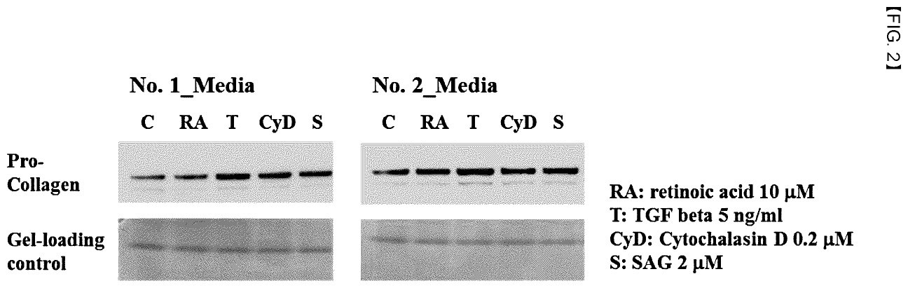

As seen from FIG. 2, when treated with the test substance, i.e., when the expression of primary cilia was increased, the amount of procollagen in fibroblasts was increased as compared to the control group (C: control group (loading control)). Both of retinoic acid (10 μM) and TGF beta (5 ng/mL), which were used as positive control groups, increased procollagen as previously known. Since procollagen is a precursor to collagen, increasing collagen through enhanced expression of procollagen may lead to skin antiaging such as improvement of skin wrinkles, enhancement of skin elasticity, enhancement of skin volume, prevention of old look, etc. Accordingly, since both cytochalasin D and SAG enhance the expression of primary cilia and the enhancement of primary cilia results in skin antiaging effect, it was confirmed that a substance capable of exerting skin antiaging effect such as improvement of skin wrinkles, enhancement of skin elasticity, enhancement of skin volume, etc. can be screened by investigating whether the expression of primary cilia is enhanced. In addition, it was confirmed that a composition containing a substance enhancing the expression of primary cilia can be used to prepare a composition for skin antiaging such as improvement of skin wrinkles, enhancement of skin elasticity, enhancement of skin volume, etc.

[Formulation Example 1] Soft Capsule

A soft capsule was prepared according to a common method by mixing 20 mg of cytochalasin D or 3-chloro-N-[trans-4-(methylamino)cyclohexyl]-N-[[3-(4-pyridinyl)phenyl]methyl]benzo[b]thiophene-2-carboxamide, 80-140 mg of L-carnitine, 180 mg of soybean oil, 2 mg of palm oil, 8 mg of hydrogenated vegetable oil, 4 mg of yellow beeswax and 6 mg of lecithin and filling the mixture in a capsule.

[Formulation Example 2] Tablet

A tablet was prepared according to a common method by mixing 20 mg of cytochalasin D or 3-chloro-N-[trans-4-(methylamino)cyclohexyl]-N-[[3-(4-pyridinyl)phenyl]methyl]benzo[b]thiophene-2-carboxamide, 200 mg of galactooligosaccharide, 60 mg of lactose and 140 mg of maltose, granulating the mixture using a fluidized-bed dryer, and then tableting the granule after adding 6 mg of sugar ester.

[Formulation Example 3] Granule

A granule was prepared by mixing 20 mg of cytochalasin D or 3-chloro-N-[trans-4-(methylam ino)cyclohexyl]-N-[[3-(4-pyridinyl)phenyl]methyl]benzo[b]thiophene-2-carboxamide, 250 mg of anhydrous crystalline glucose and 550 mg of starch, forming the mixture into a granule using a fluidized-bed granule, and filling the granule in a pouch.

[Formulation Example 4] Drink

After mixing 20 mg of cytochalasin D or 3-chloro-N-[trans-4-(methylamino)cyclohexyl]-N-[[3-(4-pyridinyl)phenyl]methyl]benzo[b]thiophene-2-carboxamide, 10 g of glucose, 0.6 g of citric acid and 25 g of oligosaccharide syrup, 300 mL of purified water was added. A drink was prepared by filling 200 mL of the resulting mixture in a bottle and then sterilizing at 130° C. for 4-5 seconds.

[Formulation Example 5] Injection

An injection was prepared according to a common method with 50 mg of cytochalasin D or 3-chloro-N-[trans-4-(methylamino)cyclohexyl]-N-[[3-(4-pyridinyl)phenyl]methyl]benzo[b]thiophene-2-carboxamide, an adequate amount of sterilized distilled water for injection and an adequate amount of a pH control agent.

[Formulation Example 6] Softening lotion (skin lotion)

A softening lotion (skin lotion) was prepared with 3.0 wt % of cytochalasin D or 3-chloro-N-[trans-4-(methylam ino)cyclohexyl]-N-[[3-(4-pyridinyl)phenyl]methyl]benzo[b]thiophene-2-carboxamide, 1.00 wt % of L-ascorbic acid 2-phosphate magnesium salt, 1.00 wt % of water-soluble collagen (1% aqueous solution), 0.10 wt % of sodium citrate, 0.05 wt % of citric acid, 0.20 wt % of licorice extract, 3.00 wt % of 1,3-bytylene glycol and purified water as balance.

[Formulation Example 7] Cream

A cream-type formulation was prepared with 3.00 wt % of cytochalasin D or 3-chloro-N-[trans-4-(methylamino)cyclohexyl]-N-[[3-(4-pyridinyl)phenyl]methyl]benzo[b]thiophene-2-carboxamide, 2.00 wt % of polyethylene glycol monostearate, 5.00 wt % of self-emulsifying glycerin monostearate, 4.00 wt % of propylene glycol, 6.00 wt % of squalene, 6.00 wt % glyceryl tri-2-ethylhexanoate, 1.00 wt % of sphingolipid, 7.00 wt % of 1,3-butylene glycol, 5.00 wt % of beeswax and purified water as balance.

[Formulation Example 8] Pack

A pack was prepared with 3.00 wt % of cytochalasin D or 3-chloro-N-[trans-4-(methylam ino)cyclohexyl]-N-[[3-(4-pyridinyl)phenyl]methyl]benzo[b]thiophene-2-carboxamide, 13.00 wt % of polyvinyl alcohol, 1.00 wt % of L-ascorbic acid 2-phosphate magnesium salt, 1.00 wt % of lauroyl hydroxyproline, 2.00 wt % of water-soluble collagen (1 aqueous solution), 3.00 wt % of 1,3-butylene glycol, 5.00 wt % of ethanol and purified water as balance.

[Formulation Example 9] Health Food

A health food was prepared according to a common method with the following ingredients.

TABLE 1

| Ingredients | Contents | |
|---|---|---|
| Cytochalasin D or | 10 | mg |
| 3-chloro-N-[trans-4-(methylamino)cyclohexyl]- | | |
| N-[[3- | | |
| (4-pyridinyl)phenyl]methyl]benzo[b]thiophene- | | |
| 2-carboxamide | | |

Vitamin mixture

| | | |
|---|---|---|
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |

Mineral mixture

| | | |
|---|---|---|
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above-described compositions of the vitamin and mineral mixtures are provided as a relatively suitable example for health food and may be varied as desired. A health food was prepared according to a common method after mixing the above ingredients.

[Formulation Example 10] Health Beverage

A health beverage was prepared according to a common method with the following ingredients.

TABLE 2

| Ingredients | Contents | |
|---|---|---|
| Cytochalasin D or | 15 | mg |
| 3-chloro-N-[trans-4-(methylamino)cyclohexyl]- | | |
| N-[[3- | | |
| (4-pyridinyl)phenyl]methyl]benzo[b]thiophene- | | |
| 2-carboxamide | | |
| Citric acid | 1000 | mg |
| Oligosaccharide | 100 | g |
| Plum extract | 2 | g |
| Taurine | 1 | g |
| Purified water | Balance | |
| Total volume | 900 | mL |

After mixing the above ingredients by adding purified water to a total volume of 900 mL, a health beverage was prepared according to a common method by heating the mixture at 85° C. for about 1 hour under stirring, filtering the resulting solution into a sterilized 2-L container, and storing in a refrigerator after sealing and sterilizing.

While the exemplary embodiments have been shown and described, it will be understood by those skilled in the art that various changes in form and details may be made thereto without departing from the spirit and scope of this disclosure as defined by the appended claims.

1. A method for skin antiaging of a subject, the method comprising: administering cytochalasin D, 3-chloro-N-[trans-4-(methylamino)cyclohexyl]-N-[[3-(4-pyridinyl)phenyl]methyl]benzo[b]thiophene-2-carboxamide, an optical isomer or stereoisomer thereof, a pharmaceutically acceptable salt thereof, a hydrate thereof or a solvate thereof to the subject as a substance increasing the expression of primary cilia.

2. The method according to claim 1, wherein the cytochalasin D is derived from a mold.

3. The method according to claim 1, wherein the mold is Zygosporium mansonii.

4. The method according to claim 1, wherein the aging is caused by procollagen reduction.

5. The method according to claim 1, wherein the skin antiaging is prevention, treatment or improvement of one or more selected from a group consisting of skin wrinkling, skin elasticity, skin regeneration, skin volume, scar and old look.

6. The method according to claim 1, wherein the composition is administered by transdermal administration or oral administration.

7. The method according to claim 1, wherein the administration dosage of the substance increasing the expression of primary cilia is 0.0001-20 mg/kg/day.

8. The method according to claim 1, wherein the substance increasing the expression of primary cilia is administered in a form of a composition, and the content of the substance increasing the expression of primary cilia is 0.0001-20 wt % based on the total weight of the composition.

9. The method according to claim 1, wherein the substance increasing the expression of primary cilia is administered in a form of a composition, and the composition is a food, pharmaceutical or cosmetic composition.

10. A method for screening a substance with skin antiaging effect, comprising:
a step of treating skin cells with a test substance; and
a step of identifying the relative degree of formation of primary cilia in the skin cells before and after the treatment with the test substance.

11. The method for screening a substance with skin antiaging effect according to claim 10, wherein the skin cells are fibroblasts.

12. The method for screening a substance with skin antiaging effect according to claim 10, which further comprises a step of determining the test substance as a substance with skin antiaging effect when the primary cilia formed after the treatment with the test substance are more than the primary cilia before the treatment.

13. The method for screening a substance with skin antiaging effect according to claim 10, wherein the relative degree of formation of primary cilia is determined through one or more of comparison of the number of the skin cells wherein primary cilia are expressed and comparison of the amount of primary cilia expressed in one cell.

14. The method for screening a substance with skin antiaging effect according to claim 10, wherein the relative degree of formation of primary cilia is identified through fluorescent staining.

15. The method for screening a substance with skin antiaging effect according to claim 10, wherein the relative degree of formation of primary cilia is identified with the degree of expression of collagen or procollagen.

16. (canceled)

17. (canceled)

18. A method for diagnosis of skin condition, comprising a step of identifying the degree of formation of primary cilia.

19. The method for diagnosis of skin condition according to claim 18, wherein the primary cilia are primary cilia of fibroblasts of the whole skin or a specific part of the skin,
wherein the degree of formation of primary cilia is identified by comparing the degree of formation of primary cilia in the whole skin or a specific part of the skin of a subject at two specific time points,
wherein a grade indicating the possibility of skin aging is evaluated depending on the degree of formation of primary cilia, and
wherein the grade is determined based on a reference value determined from data about the degree of formation of primary cilia obtained from skin samples of a given number of subjects.

20. The method for diagnosis of skin condition according to claim 18, wherein the skin condition is skin condition associated with collagen or procollagen,
wherein the degree of formation of primary cilia is identified by comparing the degree of formation of primary cilia before and after a specific stimulation inhibiting the formation of collagen or procollagen is applied, and
wherein the diagnosis of skin condition is determining whether the skin has high sensitivity to a stimulation inhibiting the formation of collagen or procollagen.

21. (canceled)

22. (canceled)

23. The method for diagnosis of skin condition according to claim 20, wherein the stimulation is one or more of exogenous stimulation and endogenous stimulation,
wherein the exogenous stimulation comprises ultraviolet radiation, physical pressure, wound, stimulation by a heavy metal, stimulation by a chemical substance, microbial infection and stress, and
wherein the endogenous stimulation comprises change of oxidative stress in vivo, aging, inflammation, hormonal change, dysfunction of a digestive organ including the liver, metabolic dysfunction, nutritional abnormality, and excess or deficiency of vitamins or minerals.

24-26. (canceled)

27. The method for diagnosis of skin condition according to claim 18, wherein it is determined that there is a possibility of developing skin aging when collagen or procollagen is decreased in the whole skin or a specific part of the skin.

28. (canceled)

29. (canceled)
